(19) United States
(12) Patent Application Publication
(10) Pub. No.: US 2020/0345684 A1
(43) Pub. Date: Nov. 5, 2020

(54) CANNABINOID COMPOSITION AND PROCESSES OF MANUFACTURE

(71) Applicant: VIALPANDO, LLC, San Francisco, CA (US)

(72) Inventors: Monica Marie VIALPANDO, San Francisco, CA (US); Danforth Parker Miller, III, San Carlos, CA (US)

(21) Appl. No.: 16/398,834

(22) Filed: Apr. 30, 2019

(51) Int. Cl. A61K 31/352; A61K 9/14

(52) U.S. Cl. CPC A61K 31/352; A61K 9/145; A61K 9/148; A61K 9/143

(57) ABSTRACT

The technology relates to compositions comprising a cannabinoid or a cannabinoid mixture adsorbed onto at least one mesoporous silica wherein the cannabinoid mixture comprises a cannabinoid and a surfactant. Preferably, the composition is in the form of a free flowing powder.

### TECHNICAL FIELD

The technology relates to compositions comprising a cannabinoid or a cannabinoid mixture adsorbed onto at least one mesoporous silica wherein the cannabinoid mixture comprises a cannabinoid and a surfactant. Preferably the composition is in the form of a free flowing powder.

### BACKGROUND

Cannabinoids are compounds derived from Cannabis sativa, an annual plant in the Cannabaceae family. The plant contains over 100 cannabinoids. The most active naturally occurring cannabinoid is tetrahydrocannabinol (THC), which is used for the treatment of a wide range of medical conditions, including glaucoma, AIDS wasting, neuropathic pain, treatment of spasticity associated with multiple sclerosis, fibromyalgia and chemotherapy-induced nausea. Additionally, THC has been reported to exhibit a therapeutic effect in the treatment of allergies, inflammation, infection, epilepsy, depression, migraine, bipolar disorders, anxiety disorder, and drug dependency and withdrawal syndromes. THC is particularly effective as an anti-emetic drug and is administered to curb emesis, a common side effect accompanying the use of opioid analgesics and anaesthetics, highly active anti-retroviral therapy and cancer chemotherapy.

Because of their hydrophobic nature, cannabinoids are poorly absorbed systemically from oral dosage forms in the aqueous environment of the gastrointestinal tract, and simple oral formulations of cannabinoids, therefore, tend to exhibit low bioavailability.

The physicochemical properties of cannabinoids, such as high lipophilicity, low aqueous solubility, high viscosity, and sensitivity to light and oxygen, present unique product formulation challenges. For example, at room temperature, these materials can be solids or viscous liquids, with the resinous or crystalline behavior depending on the particular cannabinoid, its purity, and extraction and isolation methods. Oily, viscous liquids can be particularly troublesome to formulate, process, and handle.

The primary solubility-enhancing technologies currently applied in the cannabis industry are self nano-emulsifying drug delivery technologies (SNEDDS), cyclodextrins, and liposomes. However, these technologies suffer from the disadvantage that novel or high amount of excipients are needed for solubilization and stabilization of a cannabinoid.

There is a need for processes for formulating liquid, semisolid and highly viscous materials, such as cannabinoids, into free-flowing powders.

The present inventors have developed a cannabinoid composition that exists as a free-flowing powder at room temperature. Advantageously, the compositions may improve ease of processing and flexibility for further formulation and process development. A further advantage of the composition is that they may increase the aqueous dissolution rate of the cannabinoid. The cannabinoid compositions may also enable the dissolution rate of the cannabinoid to be controlled by varying the amount or proportion of one or more of the constituents in the composition.

### SUMMARY

In a first aspect, there is provided a powder composition comprising a cannabinoid or a cannabinoid mixture adsorbed onto at least one mesoporous silica wherein the cannabinoid mixture comprises a cannabinoid and a surfactant.

The cannabinoid may be selected from the group consisting of a plant extract, cannabigerolic acid (CBGA); cannabigerolic acid monomethylether (CBGAM), cannabigerol (CBG), cannabigerol monomethylether (CBGM), cannabigerovarinic acid (CBGVA), cannabichromevarin (CBCV), cannabichromenic acid (CBCA) cannabichromene (CBC), cannabidiolic acid (CBDA), cannabidiol (CBD), cannabidiol monomethyl ether (CBDM), cannabidiol-C4 (CBD-D4), cannabidivarinic acid (CBDVA), cannabidivarin (CBDV), cannabidiorcol (CBD-D1), delta-9-tetrahydrocannabinolic acid A (THCA-A), delta-9-tetrahydrocannabinolic acid B (THCA-B), delta-9-tetrahydrocannabinol (D9-THC), delta-9-tetrahydrocannabinolic acid C4 (THCA-C4), delta-9-tetrahydrocannabinol-C4 (THC-C4), delta-9-tetrahydrocannabivarinic acid (THCVA), delta-9-tetrahydrocannabivarin (THCV), delta-9-tetrahydrocannabiorcolic acid (THCA-C1),), delta-9-tetrahydrocannabiorcol (THC-C1), delta-7-cis-iso-tetrahydrocannabivarin (D7-THCV), delta-8-tetrahydrocannabinolic (D8-THCA), delta-8-tetrahydrocannabinol (D8-THC), cannabicycloic acid (CBLA), cannabicyclol (CBL), cannabicyclovairn (CBLV), cannabielsoic acid A (CBEA-A), cannabielsoic acid B (CBEA-B), cannabielsoin (CBE), cannabinolic acid (CBNA), cannabinol (CBN), cannabinol methylether (CBNM), cannabinol-C4 (CBN-C4), cannabinol-C2 (CBN-C2), cannabivarin (CBV), cannabiorcol (CBN-C1), cannabinodiol (CBND), cannabinodivarin (CBVD), cannabitriol (CBT), 10-ethoxy-9-hydroxy-delta-6a-tetrahydrocannabinol, 8,9-dihydroxy-delta-6a-tetrahydrocannabinol, cannabitriolvarin (CBTV), ethoxy-cannabitriolvarin (CBTVE), dehydrocannabifuran (DCBG), cannabifuran (CBF), cannabichromanon (CBCN), cannabicitran (CBT), 10-oxo-delta-6a-tetrahydrocannabinol (OTHC), delta-9-cis-tetrahydrocannabinol (cis-THC), 3,4,5,6-tetrahydro-7-hydroxy-alpha-alpha-2-trimethyl-9-n-propyl-2,6-methano-2H-1-benzoxoxin-5-methanol (OH-iso-HHCV), cannabiripsol (CBR), and trihydroxy-delta-9-tetrahydrocannabinol (triOH-THC).

In one embodiment the cannabinoid is delta-9-THC or delta-8-THC.

The surfactant may be an anionic, cationic, or zwitterionic surfactant. In one embodiment the surfactant is an anionic surfactant. In a preferred embodiment, the anionic surfactant may be sodium lauryl sulfate.

In one embodiment, the concentration of the surfactant in the composition is from about 0.1% to about 35% (w/w).

The composition may further comprise a terpene or terpenoid.

The cannabinoid or cannabinoid mixture may comprise a diluent. The ratio of diluent:cannabinoid may be about 50:1 to about 1:50.

In some embodiments the diluent may be a plant-based oil such as a vegetable oil.

The mesoporous silica may be ordered mesoporous silica or disordered mesoporous silica.

In some embodiments the mesoporous silica has an average pore volume of about 0.50 cm³/g to about 10 cm³/g. The mesoporous silica may have an average pore size of about 2 nm to about 50 nm.

In some embodiments the mesoporous silica may be a mesoporous silica particle. The mesoporous silica particles have an average particle size diameter of between about 2 μm to at least about 250 μm, for example the average diameter may be about 2 μm, about 10 μm, about 25 μm, about 50 μm, about 75 μm, about 100 μm, about 125 μm, about 150 μm, about 175 μm, about 200 μm, about 225 μm, or at least about 250 μm.

The ratio of surfactant:mesoporous silica may be from about 1:5 to about 1:50. In one embodiment the ratio of surfactant:mesoporous silica may be from about 1:25 to about 1:35, for example about 1:29.

In one embodiment, the composition is a flowable powder.

In one embodiment the composition comprises a blend of two or more mesoporous silica.

In a second aspect there is provided a formulation comprising an effective amount of the composition of the first aspect and at least one carrier, diluent or excipient. The excipient may be one or more of microcrystalline cellulose, croscarmellose sodium, and magnesium stearate. In preferred embodiments the formulation is a pharmaceutically acceptable formulation.

In a third aspect there is provided a process of preparing the composition of the first aspect, comprising

a) heating the cannabinoid;

b) mixing the cannabinoid and the mesoporous silica, wherein the cannabinoid adsorbs to the mesoporous silica.

In one embodiment step b) further comprises mixing the cannabinoid with the surfactant wherein the cannabinoid and the surfactant form a cannabinoid mixture that adsorbs to the mesoporous silica.

In a fourth aspect there is provided a process of preparing the composition of the first aspect, comprising

a) heating the cannabinoid;

b) mixing the cannabinoid with the surfactant wherein the cannabinoid and the surfactant form a cannabinoid mixture;

c) mixing the cannabinoid mixture and the mesoporous silica, wherein the cannabinoid mixture adsorbs to the mesoporous silica.

In a fifth aspect there is provided a process of preparing the composition of the first aspect, comprising

a) heating the surfactant;

b) mixing the cannabinoid with the surfactant wherein the cannabinoid and the surfactant form a cannabinoid mixture;

c) mixing the cannabinoid mixture and the mesoporous silica, wherein the cannabinoid mixture adsorbs to the mesoporous silica.

In a sixth aspect there is provided a process of preparing the composition of the first aspect, comprising

a) mixing the cannabinoid with the surfactant wherein the cannabinoid and the surfactant form a cannabinoid mixture;

b) heating the cannabinoid mixture;

c) mixing the cannabinoid mixture and the mesoporous silica, wherein the cannabinoid mixture adsorbs to the mesoporous silica.

In embodiments, the cannabinoid or cannabinoid mixture is heated to a temperature that increases fluidity or decreases viscosity. For example the cannabinoid or cannabinoid mixture may be heated to a temperature up to about 100° C.

In embodiments where the cannabinoid is crystalline at room temperature, it is heated above its melting temperature. For example the cannabinoid may be heated to about 20° C. above its melting temperature.

In embodiments where the cannabinoid is resinous at room temperature, it is heated above its glass transition temperature. For example the cannabinoid may be heated to about 20° C. above its glass transition temperature.

The process may further comprise the step of stirring the cannabinoid or cannabinoid mixture and the mesoporous silica.

In a seventh aspect there is provided a food, beverage or cosmetic product comprising the composition of the first aspect.

In an eighth aspect there is provided a method of treatment of a disease or condition responsive to a cannabinoid, the method comprising administering to the subject an effective amount of a composition of the first aspect or a formulation of the second aspect.

In a ninth aspect there is provided use of a composition of the first aspect for the manufacture of a medicament for treatment of a disease or condition responsive to a cannabinoid.

In a tenth aspect there is provided a composition of the first aspect for use in treatment of a disease or condition responsive to a cannabinoid.

The disease or condition may be selected from the group comprising pain, spasticity associated with multiple sclerosis, nausea, posttraumatic stress disorder, cancer, epilepsy, cachexia, glaucoma, HIV/AIDS, degenerative neurological conditions, anorexia and weight loss associated with HIV, irritable bowel syndrome, epilepsy, spasticity, Tourette syndrome, amyotrophic lateral sclerosis, Huntington's disease, Parkinson's disease, dystonia, dementia, glaucoma, traumatic brain injury, addiction, anxiety, depression, sleep disorders, posttraumatic stress disorder, and schizophrenia.

### Definitions

Throughout this specification, unless the context clearly requires otherwise, the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

Throughout this specification, the term “consisting of” means consisting only of.

Throughout this specification, the term “consisting essentially of” means the inclusion of the stated element(s), integer(s) or step(s), but other element(s), integer(s) or step(s) that do not materially alter or contribute to the working of the invention may also be included.

Any discussion of documents, acts, materials, devices, articles or the like which has been included in the present specification is solely for the purpose of providing a context for the present technology. It is not to be taken as an admission that any or all of these matters form part of the prior art base or were common general knowledge in the field relevant to the present technology as it existed before the priority date of each claim of this specification.

Unless the context requires otherwise or specifically stated to the contrary, integers, steps, or elements of the technology recited herein as singular integers, steps or elements clearly encompass both singular and plural forms of the recited integers, steps or elements.

In the context of the present specification the terms “a” and “an” are used to refer to one or more than one (i.e., at least one) of the grammatical object of the article. By way of example, reference to “an element” means one element, or more than one element.

In the context of the present specification the term “about” means that reference to a FIGURE or value is not to be taken as an absolute FIGURE or value, but includes margins of variation above or below the FIGURE or value in line with what a skilled person would understand according to the art, including within typical margins of error or instrument limitation. In other words, use of the term “about” is understood to refer to a range or approximation that a person or skilled in the art would consider to be equivalent to a recited value in the context of achieving the same function or result.

Those skilled in the art will appreciate that the technology described herein is susceptible to variations and modifications other than those specifically described. It is to be understood that the technology includes all such variations and modifications. For the avoidance of doubt, the technology also includes all of the steps, features, and compounds referred to or indicated in this specification, individually or collectively, and any and all combinations of any two or more of said steps, features and compounds.

The term “effective amount” refers to an amount of a cannabinoid sufficient to produce a desired therapeutic, pharmacological, or physiological effect in the subject being treated. The term is intended to qualify the amount of the cannabinoid that will achieve the goal of improvement in disease severity and/or the frequency of incidence over treatment of each agent by itself while preferably avoiding or minimizing adverse side effects. Those skilled in the art can determine an effective dose using information and routine methods known in the art.

As used herein the terms “adsorbed”, “adsorbed to”, “adsorbed onto” and “adsorbed” are equivalent and are used interchangeably. In one or more embodiments, adsorption may comprise the cannabinoid mixture being adsorbed into the volume or bulk of the mesoporous silica. In other embodiments, adsorption of the cannabinoid mixture to the surface of the mesoporous silica may be by way of intermolecular forces between the cannabinoid mixture and the mesoporous silica.

A “carrier, diluent or excipient” includes, but is not limited to, any medium comprising a suitable water soluble organic carrier, conventional solvents, oil, hydrophobic diluent, dispersion media, fillers, solid carriers, coatings, antibacterial and antifungal agents, isotonic and absorption delaying agents. Suitable water-soluble organic carriers include, but are not limited to, saline, dextrose, corn oil, dimethylsulfoxide, and gelatin or hydroxypropylmethylcellulose capsules. Other conventional additives include lactose, mannitol, corn starch, potato starch, binders such as microcrystalline cellulose, cellulose derivatives such as hydroxypropylmethylcellulose, acacia, gelatins, disintegrators such as sodium carboxymethylcellulose, and lubricants such as talc or magnesium stearate.

“Subject” includes any human or non-human mammal. Thus, in addition to being useful for human treatment, the compounds of the present invention may also be useful for veterinary treatment of mammals, including companion animals and farm animals, such as, but not limited to dogs, cats, horses, cows, sheep, and pigs. In preferred embodiments the subject is a human.

In the context of this specification the term “administering” and variations of that term including “administer” and “administration”, includes contacting, applying, delivering or providing a compound or composition of the invention to a subject by any appropriate means.

### DESCRIPTION OF EMBODIMENTS

The compositions disclosed herein comprise a mixture of a cannabinoid (either purified or as part of a plant extract) and a surfactant which is adsorbed onto a mesoporous silica. In some embodiments the compositions disclosed herein comprise a cannabinoid (either purified or as part of a plant extract) which is adsorbed onto a mesoporous silica.

The technology described herein can provide any one or more of a number of advantages. For example, in some embodiments the technology is capable of achieving a higher cannabinoid load compared to other solubility enhancing technologies, due to the high specific surface area (˜700 m²/g) and large pore volume (˜1 cm³/g) of the mesoporous silica. In some embodiments described herein, the interaction between the mesoporous silica and the cannabinoid mixture is not critical for loading and stability, making the technology suitable for a wide range of cannabinoids and plant extracts containing cannabinoids.

### Mesoporous Silica

Mesoporous silica is a solid, highly porous material. The nanometer-scale pores result in extremely high specific surface areas. As described herein, adsorption of a mixture of a cannabinoid or cannabis extract and a surfactant on mesoporous silica converts a viscous liquid into a free-flowing powder due to the extremely high specific surface area of the silica structure. This improves the flow properties when compared to that of the cannabinoid or cannabis extract alone and is advantageous due to ease of processing for downstream development. In addition to its process improvement capabilities, mesoporous silica can enhance the aqueous solubility of cannabinoids, especially for cannabinoids that are crystalline at room temperature. In particular, the crystal structure is disrupted and the amorphous form of the drug is confined in the pore structure. This results in a higher apparent solubility and dissolution rate when compared to the crystalline form.

Although the solubility of resinous cannabinoids is enhanced due to the distribution of the drug across the large specific surface area of the mesoporous silica, the incorporation of solubility enhancing excipients may further enhance the dissolution rate and solubility of the cannabinoid. Accordingly, a further advantage of embodiments of the technology is that may be used to control and modify the release rate of cannabinoid compounds, which is a key attribute for obtaining desired drug release properties. In one embodiment, release rate may be controlled or modified based on pore size. In other embodiments, the choice of surfactant can be used to modify release rate.

Mesoporous silica exhibits excellent thermostability properties, making it an excellent material to preserve the physicochemical stability of the cannabis extract during processing and storage, which is especially beneficial for cannabis extracts comprising volatile terpenes and terpenoids. In particular, adsorption of the cannabinoid mixture onto mesoporous silica reduces the volatility of the terpenes and terpenoids, thereby reducing evaporative losses of these compounds. Moreover, mesoporous silica is biologically inert and biocompatible. This is in contrast to alternative technologies that use cyclodextrins, novel excipients or large amounts of excipients to solubilise and stabilise an active (e.g., SNEDDS, solid dispersions).

Any of several variants of mesoporous silica can be used to prepare the compositions of the invention. Pharmaceutical grade mesoporous silica is typically prepared by a sol-gel process, producing either a disordered mesoporous structure (DMS) or ordered mesoporous structure (OMS) pore structure. Both are available in a wide range of particle sizes, specific surface areas, and pore volumes, making them applicable for a variety of cannabinoids and drug delivery approaches. DMS is commercially available and used in the pharmaceutical, cosmetic, food, and beverage industries for a wide variety of applications.

DMS is commercially available and is comprised of a coherent and rigid network of continuous pores. DMS may be manufactured by any known means. In some embodiments, DMS may be synthesized via sol-gel chemistry where the particle characteristics are produced into this highly porous material.

Ordered Mesoporous Silicas (OMS) were first synthesized as molecular sieves and are now applied to a variety of fields such as adsorption, chromatography, catalysis, and optics. As with DMS, the mesopore structure is synthesized via sol-gel synthesis but utilizes a template such as surfactant or polymeric micelles to control pore structure. After the silica is polymerized, the template is removed, leading to its porosity and narrow pore size distribution. It should be noted that they are referred to as “ordered” despite their amorphous walls. Examples of OMS material types are MCM-41 and SBA-15, which form a hexagonal porous structure.

Silica is “Generally Recognized As Safe” by the United States Food and Drug Administration (FDA). Recently, silica nanoparticles in the form of Cornell dots (C dots) received FDA approval for a Phase I human clinical trial for targeted molecular imaging. It was reported that mesoporous silica exhibited a three-stage degradation behavior in simulated body fluid, suggesting that MSNs might degrade after administration, which is favorable for cargo release. Several in vivo biodistribution studies of MSNs have been reported. One study evaluated the systematic toxicity of MSNs after intravenous injection of single and repeated dose to mice. The results of clinical features, pathological examinations, mortalities, and blood biochemical indices indicated low in vivo toxicity of MSNs. It was also reported that MSNs were mainly excreted through feces and urine following different administration routes.

According to the International Union of Pure and Applied Chemistry (IUPAC), pore sizes in mesoporous silica are in the range of 2-50 nm and an ordered arrangement of pores. The pore size of the mesoporous silica can be controlled during production. The pore volume may be about 0.5 cm³/g, 1 cm³/g, 2 cm³/g, 3 cm³/g, 4 cm³/g, 5 cm³/g, 6 cm³/g, 7 cm³/g, 8 cm³/g, 9 cm³/g, or about 10 cm³/g. In some embodiments, the pore volume is around 2 cm³/g when the pore size is less than 15 nm and surface area is about 1000 m²/g.

The interaction of cannabinoid with mesopores is a surface phenomenon. The amount of cannabinoid mixture adsorbed can be determined by changes in pore volume. In ordered mesoporous material, many consecutive loadings of the cannabinoid mixture can result in almost complete filling of mesopores, indicating that the amount of cannabinoid is directly proportional to pore volume. That is, while a greater pore volume will enable a greater cannabinoid loading, the remaining pore volume will decrease with the amount of drug loaded.

For both DMS and OMS the surface area of the mesoporous silica is a determining factor for the quantity of adsorbed cannabinoids, although it is believed that surface chemistry may also be influential. To control the amount of incorporated cannabinoid mixture in the matrix, two different approaches are used, namely modifying (increasing or decreasing) the surface area and modifying the affinity of the surface for the cannabinoid. The amount of cannabinoid mixture (or other drug) adsorbed is directly proportional to specific surface area. For example, MCM-41 is synthesized by specific surface area (SBET value) 1157 m²g⁻¹ and SBA-15 with specific surface area value of 719 m²g⁻¹. For example, when alendronate is loaded in mesoporous silica particles under same conditions, 139 mg·g⁻¹ of drug is loaded in MCM-41 while 83 mg·g⁻¹ in SBA-15. This indicates that specific surface area value is closely related to the maximum loading of the drug.

In some embodiments the mesoporous silica is a particle having an average diameter from 2-250 μm.

The mesoporous silica particles may have an average diameter of between about 2 μm to at least about 250 μm, for example the average diameter may be about 2 μm, about 10 μm, about 25 μm, about 50 μm, about 75 μm, about 100 μm, about 125 μm, about 150 μm, about 175 μm, about 200 μm, about 225 μm, or at least about 250 μm.

The structural characteristics of some mesoporous silica suitable for use in the invention are listed in Table 1.

TABLE 1
Characteristics of selected mesoporous silica

| | | Pore Size | Pore Volume |
|---|---|---|---|
| Name | Pore Symmetry | (nm) | (cm³/g) |
| MCM-41 | 2D hexagonal P6mm | 1.5-8 | >1.0 |
| MCM-48 | 3D cubic Ia3d | 2-5 | >1.0 |
| MCM-50 | Lamellar p2 | 2-5 | >1.0 |
| SBA-11 | 3D cubic Pm3m | 2.1-3.6 | 0.68 |
| SBA-12 | 3D hexagonal | 3.1 | 0.83 |
| | P6₃/mmc | | |
| SBA-15 | 2D hexagonal p6mm | 6-12 | 1.17 |
| SBA-16 | Cubic Im3m | 5-15 | 0.91 |
| KIT-5 | Cubic Fm3m | 9.3 | 0.45 |
| COK-12 | 2D Hexagonal P6mm | 6-12 | 1.17 |

Other mesoporous silicas may be used in the compositions and formulations of the invention, including for example FSM-16, which has folded sheets of mesoporous materials. Various other commercially available mesoporous silica products can be used including those developed by Technical Delft University (TUD-1), Hiroshima Mesoporous Material-33 (HMM-33), Centrum voor Oppervlaktechemie en Katalyse/Centre for Research Chemistry and Catalysis (COK-12), all of which vary in their pore symmetry and shape.

In some embodiments SYLOID® 63FP/AL-1, SYLOID® 72FP SYLOID® 244FP, SYLOID® XDP 3050, SYLOID® XDP 3150, may also be used. In a preferred embodiment the mesoporous silica is SYLOID® 3050 XDP. The characteristics of the Syloid mesoporous silicas are presented in Table 2.

TABLE 2
Syloid ® silica

| | | | SY- | | SYLOID ® |
|---|---|---|---|---|---|
| Pro- | SYLOID ® | SYLOID ® | LOID ® | SYLOID ® | XDP |
| perty | 63FP/AL-1 | 72FP | 244FP | XDP 3050 | 3150 |
| Avg | 7.5 | 6.0 | 3.5 | 50 | 150 |
| par- | | | | | |
| ticle | | | | | |
| size | | | | | |
| (μm) | | | | | |
| Avg | 0.4 | 1.2 | 1.6 | 1.7 | 1.7 |
| Pore | | | | | |
| Vol- | | | | | |
| ume | | | | | |
| (cm³/ | | | | | |
| g) | | | | | |

In other embodiments fumed silica (such as Aeropearl® by evonik) and magnesium aluminium silica (for example Neuselin®) may be used.

### Cannabinoids

The cannabinoid can be synthetic or a naturally occurring cannabinoid derived from a plant. Typically, the plant is of the genus Cannabis. Cannabinoids that occur in other plant genera can also be used in the formulations. For example, cannabinoids derived from plants of the genera Echinacea, Acmella, Helichrysum, and Radula can be used in the compositions. For example, the lipophilic alkamides (alkylamides) from Echinacea species including the cis/trans isomers dodeca-2E,4E,8Z,10E/Z-tetraenoic-acid-isobutylamide can be used. Other suitable cannabinoids include beta-caryophyllene and anandamide.

Cannabinoid compounds suitable for use in the invention include, but are not limited to, tetrahydrocannabinoids, their precursors, alkyl (particularly propyl) analogues, cannabidiols, their precursors, alkyl (particularly propyl) analogues,

The cannabinoid may be selected from the group consisting of: cannabigerolic acid (CBGA); cannabigerolic acid monomethyl ether (CBGAM), cannabigerol (CBG), cannabigerol monomethyl ether (CBGM), cannabigerovarinic acid (CBGVA), cannabichromevarin (CBCV), cannabichromenic acid (CBCA) cannabichromene (CBC), cannabidiolic acid (CBDA), cannabidiol (CBD), cannabidiol monomethyl ether (CBDM), cannabidiol-C4 (CBD-D4), cannabidivarinic acid (CBDVA), cannabidivarin (CBDV), cannabidiorcol (CBD-D1), delta-9-tetrahydrocannabinolic acid A (THCA-A), delta-9-tetrahydrocannabinolic acid B (THCA-B), delta-9-tetrahydrocannabinol (D9-THC), delta-9-tetrahydrocannabinolic acid C4 (THCA-C4), delta-9-tetrahydrocannabinol-C4 (THC-C4), delta-9-tetrahydrocannabivarinic acid (THCVA), delta-9-tetrahydrocannabivarin (THCV), delta-9-tetrahydrocannabiorcolic acid (THCA-C1),), delta-9-tetrahydrocannabiorcol (THC-C1), delta-7-cis-iso-tetrahydrocannabivarin (D7-THCV), delta-8-tetrahydrocannabinolic (D8-THCA), delta-8-tetrahydrocannabinol (D8-THC), cannabicycloic acid (CBLA), cannabicyclol (CBL), cannabicyclovairn (CBLV), cannabielsoic acid A (CBEA-A), cannabielsoic acid B (CBEA-B), cannabielsoin (CBE), cannabinolic acid (CBNA), cannabinol (CBN), cannabinol methylether (CBNM), cannabinol-C4 (CBN-C4), cannabinol-C2 (CBN-C2), cannabivarin (CBV), cannabiorcol (CBN-C1), cannabinodiol (CBND), cannabinodivarin (CBVD), cannabitriol (CBT), 10-ethoxy-9-hydroxy-delta-6a-tetrahydrocannabinol, 8,9-dihydroxy-delta-6a-tetrahydrocannabinol, cannabitriolvarin (CBTV), ethoxy-cannabitriolvarin (CBTVE), dehydrocannabifuran (DCBG), cannabifuran (CBF), cannabichromanon (CBCN), cannabicitran (CBT), 10-oxo-delta-6a-tetrahydrocannabinol (OTHC), delta-9-cis-tetrahydrocannabinol (cis-THC), 3,4,5,6-tetrahydro-7-hydroxy-alpha-alpha-2-trimethyl-9-n-propyl-2,6-methano-2H-1-benzoxoxin-5-methanol (OH-iso-HHCV), cannabiripsol (CBR), and trihydroxy-delta-9-tetrahydrocannabinol (triOH-THC), cannabichromene, cannabichromene propyl analogue, ajulemic acid, cannabinor, and any combination of two or more of these cannabinoids.

In some embodiments the cannabinoid may be present in an extract of a plant. Accordingly, ‘cannabinoid mixtures’ as used herein includes mixtures containing two or more cannabinoids, including plant extracts comprising a mixture of two or more cannabinoids. For example the silicas may be two different types of silica (e.g., Syloid 244 and Syloid 3050) or two or more portions of the same type of silica each with a different particle size distribution.

Plant extracts containing cannabinoids may also contain one or more terpenes and/or terpenoids. For example the plant extracts may contain a terpene selected from the group comprising t-carophyllene, myrcene, α-humulene, α-pinene, α-bisabolol, β-pinene, limonene, ocimene and/or terpinolene, guaiol, α-terpineol, and terpinolene, linalool, fenchol, guaiene, and 3-careen. Accordingly, “cannabinoid mixtures” as used herein may contain one or more terpenes.

The cannabinoid or cannabinoid mixture may be present in any amount suitable for a desired application. For example, the cannabinoid or plant extract containing the cannabinoid may be present in an amount ranging from less than about 1% to about 90 weight %, relative to the weight of the composition. A higher or lower concentration of the cannabinoid mixture may be used, and the concentration may vary within the aforementioned range. For example, the cannabinoid may be present in an amount ranging from about 0.01% to about 50%, about 1% to about 50%, about 2 to about 5%, about 5% to about 10%, about 10% to about 20%, about 20% to about 30%, about 30% to about 40%, or about 40% to about 50% by weight of the formulation. In some embodiments, the cannabinoid may be present in an amount ranging from about 25% to about 30%, about 30% to about 35%, or about 35% to about 40% by weight of the formulation. In some embodiments a desired amount of cannabinoid or cannabinoid mixture may be achieved by repeatedly loading the mesoporous silica with the cannabinoid or the cannabinoid mixture.

### Surfactants

In some embodiments the compositions of the invention comprise a surfactant to improve loading of the cannabinoid onto the mesoporous silica. The surfactant also facilitates improved desorption of the cannabinoid from the mesoporous silica into aqueous solution and/or desorption of the cannabinoid. In some embodiments, the cannabinoid and the surfactant are mixed to form a cannabinoid mixture prior to adsorption (loading) on to a mesoporous silica. In other embodiments, the cannabinoid, surfactant and the mesoporous silica are mixed together and the cannabinoid mixture forms concomitantly with loading.

Surfactants play important roles in the compositions. First, a surfactant lowers the surface tension of a liquid. This facilitates loading solution-based drugs into nano-sized pores. Second, through reduction of surface tension, surfactants facilitate (in vivo) wetting of the finished dosage form. This is an important step in dissolution of the drug and helps increase the delivery of the drug from the dosage form.

In some embodiments the surfactant is an anionic surfactant. Suitable anionic surfactants include alkyl sulfonates, aryl sulfonates, alkyl phosphates, alkyl phosphonates, potassium laurate, sodium lauryl sulfate, sodium dodecylsulfate, alkyl polyoxyethylene sulfates, sodium alginate, dioctyl sodium sulfosuccinate, phosphatidic acid and their salts, sodium carboxymethylcellulose, bile acids and their salts, cholic acid, deoxycholic acid, glycocholic acid, taurocholic acid, and glycodeoxycholic acid, and calcium carboxymethylcellulose, stearic acid and its salts, (e.g., calcium stearate), phosphates, sodium dodecylsulfate, carboxymethylcellulose calcium, carboxymethylcellulose sodium, dioctylsulfosuccinate, dialkylesters of sodium sulfosuccinic acid, diethanolamine lauryl sulfate, sodium lauryl sulfate and phospholipids. A preferred surfactant is sodium lauryl sulfate or sodium dodecylsulfate.

In some embodiments the surfactant is a cationic surfactant. Suitable cationic surfactants include quaternary ammonium compounds, benzalkonium chloride, cetyltrimethylammonium bromide, chitosans, lauryldimethylbenzylammonium chloride, acyl carnitine hydrochlorides, alkyl pyridinium halides, cetyl pyridinium chloride, cationic lipids, polymethylmethacrylate trimethylanmonium bromide, sulfonium compounds, polyvinylpyrrolidone-2-dimethylaminoethyl methacrylate dimethyl sulfate, hexadecyltrimethyl ammonium bromide, phosphonium compounds, quaternary ammonium compounds, benzyl-di(2-chloroethyl)ethylammonium bromide, coconut trimethyl ammonium chloride, coconut trimethyl ammonium bromide, coconut methyl dihydroxyethyl ammonium chloride, coconut methyl dihydroxyethyl ammonium bromide, decyl triethyl ammonium chloride, decyl dimethyl hydroxyethyl ammonium chloride, decyl dimethyl hydroxyethyl ammonium chloride bromide, C12-15-dimethyl hydroxyethyl ammonium chloride, C12-15-dimethyl hydroxyethyl ammonium chloride bromide, coconut dimethyl hydroxyethyl ammonium chloride, coconut dimethyl hydroxyethyl ammonium bromide, myristyl trimethyl ammonium methyl sulfate, lauryl dimethyl benzyl ammonium chloride, lauryl dimethyl benzyl ammonium bromide, lauryl dimethyl (ethenoxy)4 ammonium chloride, lauryl dimethyl (ethenoxy)4 ammonium bromide, N-alkyl (C12-18)dimethylbenzyl ammonium chloride, N-alkyl (C14-18)dimethylbenzyl ammonium chloride, N-tetradecylidmethylbenzyl ammonium chloride monohydrate, dimethyl didecyl ammonium chloride, N-alkyl and (C12-14) dimethyl 1-napthylmethyl ammonium chloride, trimethylammonium halide alkyl-trimethylammonium salts, dialkyl-dimethylammonium salts, lauryl trimethyl ammonium chloride, ethoxylated alkyamidoalkyldialkylammonium salts, ethoxylated trialkyl ammonium salts, dialkylbenzene dialkylammonium chloride, N-didecyldimethyl ammonium chloride, N-tetradecyldimethylbenzyl ammonium chloride monohydrate, N-alkyl(C12-14) dimethyl 1-naphthylmethyl ammonium chloride, dodecyldimethylbenzyl ammonium chloride, dialkyl benzenealkyl ammonium chloride, lauryl trimethyl ammonium chloride, alkylbenzyl methyl ammonium chloride, alkyl benzyl dimethyl ammonium bromide, C12 trimethyl ammonium bromides, C15trimethyl ammonium bromides, C17 trimethyl ammonium bromides, dodecylbenzyl triethyl ammonium chloride, poly-diallyldimethylammonium chloride (DADMAC), dimethyl ammonium chlorides, alkyldimethylammonium halogenides, tricetyl methyl ammonium chloride, decyltrimethylammonium bromide, dodecyltriethylammonium bromide, tetradecyltrimethylammonium bromide, methyl trioctylammonium chloride, “POLYQUAT 10” (a mixture of polymeric quartenary ammonium compounds), tetrabutylammonium bromide, benzyl trimethylammonium bromide, choline esters, benzalkonium chloride, stearalkonium chloride, cetyl pyridinium bromide, cetyl pyridinium chloride, halide salts of quaternized polyoxyethylalkylamines, “MIRAPOL,” (polyquaternium-2) “ALKAQUAT”, alkyl pyridinium salts, amines, amine salts, imide azolinium salts, protonated quaternary acrylamides, methylated quaternary polymers, and cationic guar gum, benzalkonium chloride, dodecyl trimethyl ammonium bromide, triethanolamine, and poloxamines.

In some embodiments the surfactant is a nonionic surfactant. Suitable nonionic surfactants include polyoxyethylene fatty alcohol ethers, polyoxyethylene sorbitan fatty acid esters, polyoxyethylene fatty acid esters, sorbitan esters, glyceryl esters, glycerol monostearate, polyethylene glycols, polypropylene glycols, polypropylene glycol esters, cetyl alcohol, cetostearyl alcohol, stearyl alcohol, aryl alkyl polyether alcohols, polyoxyethylene-polyoxypropylene copolymers, poloxamers, poloxamines, methylcellulose, hydroxycellulose, hydroxymethylcellulose, hydroxypropylcellulose, hydroxypropylinethylcellulose, noncrystalline cellulose, polysaccharides, starch, starch derivatives, hydroxyethylstarch, polyvinyl alcohol, polyvinylpyrrolidone, triethanolamine stearate, amine oxides, dextran, glycerol, gum acacia, cholesterol, tragacanth, glycerol monostearate, cetostearyl alcohol, cetomacrogol emulsifying wax, sorbitan esters, polyoxyethylene alkyl ethers, polyoxyethylene castor oil derivatives, polyoxyethylene sorbitan fatty acid esters, polyethylene glycols, polyoxyethylene stearates, hydroxypropyl celluloses, hydroxypropyl methylcellulose, methylcellulose, hydroxyethylcellulose, hydroxypropylmethylcellulose phthalate, noncrystalline cellulose, polyvinyl alcohol, polyvinylpyrrolidone, 4-(1,1,3,3-tetramethylbutyl)phenol polymer with ethylene oxide and formaldehyde, poloxamers, alkyl aryl polyether sulfonates, mixtures of sucrose stearate and sucrose distearate, C₁₈H₃₇CH₂C(O)N(CH₃)CH₂(CHOH)₄(CH₂OH)₂, p-isononylphenoxypoly(glycidol), decanoyl-N-methylglucamide, n-decyl-β-D-glucopyranoside, n-decyl-β-D-maltopyranoside, n-dodecyl-β-D-glucopyranoside, n-dodecyl-β-D-maltoside, heptanoyl-N-methylglucamide, n-heptyl-β-D-glucopyranoside, n-heptyl-β-D-thioglucoside, n-hexyl-β-D-glucopyranoside; nonanoyl-N-methylglucamide, n-nonyl-β-D-glucopyranoside, octanoyl-N-methylglucamide, n-octyl-β-D-glucopyranoside, octyl-β-D-thioglucopyranoside, PEG-cholesterol, PEG-cholesterol derivatives, PEG-vitamin A, PEG-vitamin E, and random copolymers of vinyl acetate and vinyl pyrrolidone.

In some embodiments the surfactant is a zwitterionic surfactant. Suitable zwitterionic surfactants include zwitterionic phospholipids, for example phosphatidylcholine, phosphatidylethanolamine, diacyl-glycero-phosphoethanolamine (such as dimyristoyl-glycero-phosphoethanolamine (DMPE), dipalmitoyl-glycero-phosphoethanolamine (DPPE), distearoyl-glycero-phosphoethanolamine (DSPE), and dioleolyl-glycero-phosphoethanolamine (DOPE)). Mixtures of phospholipids that include anionic and zwitterionic phospholipids may be employed in this invention. Such mixtures include but are not limited to lysophospholipids, egg or soybean phospholipid or any combination thereof.

In preferred embodiments the surfactant is sodium lauryl sulfate.

The ratio of surfactant:mesoporous silica can be used to modulate adsorption of the cannabinoid onto the mesoporous silica. Similarly, the ratio of surfactant:mesoporous silica can be used to modulate desorption of the cannabinoid.

In some embodiments, the ratio of surfactant:cannabinoid is between about 1:1 to about 1:50. For example the ratio may be about 1:5, 1:6, 1:7, 1:8, 1:9, 1:10, 1:11, 1:12, 1:13, 1:14, 1:15, 1:16, 1:17, 1:18, 1:19, 1:20, 1:21, 1:22, 1:23, 1:24, 1:25, 1:26, 1:27, 1:28, 1:29, 1:30, 1:31, 1:32, 1:33, 1:34, 1:35, 1:36, 1:37, 1:38, 1:39, 1:40, 1:41, 1:42, 1:43, 1:44, 1:45, 1:46, 1:47, 1:48, 1:49, or about 1:50.

In one embodiment, the mass ratio of surfactant:mesoporous silica is from about 1:25 to about 1:35, for example 1:29 which in an exemplary formulation corresponds to about 1.5% w/w surfactant and about 43% (w/w) mesoporous silica.

In preferred embodiments, the mass ratio of surfactant:mesoporous silica is about 1:29.

### Diluents

The cannabinoid may be diluted with a suitable diluent. Dilution may be desired for example to achieve a desired dosage of the cannabinoid in the composition or to facilitate ease of handling of the cannabinoid prior to incorporation into the composition. Alternatively or in addition, dilution may be used to impart other desirable characteristics such as flavour or aroma to the composition. Alternatively or in addition dilution may be used to mask undesirable taste or smell.

In some embodiments, the ratio of diluent:cannabinoid is between about 1:1 to about 1:50. For example the ratio may be about 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:21, 1:22, 1:23, 1:24, 1:25, 1:26, 1:27, 1:28, 1:29, 1:30, 1:31, 1:32, 1:33, 1:34, 1:35, 1:36, 1:37, 1:38, 1:39, 1:40, 1:41, 1:42, 1:43, 1:44, 1:45, 1:46, 1:47, 1:48, 1:49, or about 1:50.

In other embodiments the ratio of diluent:cannabinoid is between about 50:1 to about 1:1. For example the ratio may be about 50:1, 1:49, 1:48, 1:47, 1:46, 1:45, 1:44, 1:43, 1:42, 1:41, 1:40, 1:39, 1:38, 1:37, 1:36, 1:35, 1:34, 1:33, 1:32, 1:31, 1:30, 1:29, 1:28, 1:27, 1:26, 1:25, 1:24, 1:23, 1:22, 1:21, 1:20, 1:19, 1:18, 1:17, 1:16, 1:15, 1:14, 1:13, 1:12, 1:11, 1:10, 1:9, 1:8, 1:7, 1:6, 1:5, 1:4, 1:3, 1:2, or about 1:1.

Suitable diluents include oils and waxes that are known to be safe for administration to a subject. For example, suitable diluents may be mineral oils, vegetable oils, fluorinated or perfluorinated oils, natural or synthetic waxes, silicones, cationic polymers, proteins and hydrolyzed proteins, ceramide type compounds, fatty amines, fatty acids and their derivatives, as well as mixtures of these different compounds.

The synthetic oils include polyolefins, e.g., poly-α-olefins such as polybutenes, polyisobutenes and polydecenes.

The mineral oils suitable for use in the compositions of the invention include hexadecane and oil of paraffin.

Animal and vegetable oils may be used as diluents including oil from olive, sunflower, safflower, canola, corn, soy, avocado, jojoba, squash, raisin seed, sesame seed, nuts (for example peanut, walnut, hazelnut, etc.), fish, eucalyptus, lavender, vetiver, litsea cubeba, lemon, sandalwood, rosemary, chamomile, savory, nutmeg, cinnamon, hyssop, caraway, orange, geranium, cade, bergamot, glycerol tricaprocaprylate, purcellin oil, mint oil (e.g., peppermint, spearmint) and blends thereof.

Natural or synthetic waxes may also be used as diluents, these include carnauba wax, candelila wax, alfa wax, paraffin wax, ozokerite wax, vegetable waxes such as olive wax, rice wax, hydrogenated jojoba wax, absolute flower waxes such as black currant flower wax, animal waxes such as bees wax, modified bees wax (cerabellina), marine waxes and polyolefin waxes such as polyethylene wax, and blends thereof.

### Cannabinoid Mixture

Preparation of the cannabinoid mixture involves the addition of the surfactant to the purified cannabinoid or plant extract. In an alternative embodiment, the cannabinoid is added to surfactant. In one embodiment this may occur as a separate step prior to loading the mesoporous silica with the mixture. In other embodiments the surfactant, purified cannabinoid or plant extract and the mesoporous silica are combined in a single step and the cannabinoid mixture is formed concomitantly with loading.

In preferred embodiments, it may be advantageous to use a concentration of surfactant that is at or near the CMC (critical micelle concentration). In other embodiments the surfactant concentration may be in excess of the CMC so that at least a portion of the cannabinoid is contained in micelles of the surfactant.

Accordingly, the ratio of surfactant:cannabinoid is between about 1:1000 to about 1:5. For example the ratio may be about 1:1000, 1:950, 1:900, 1:850, 1:800, 1:750, 1:700, 1:650, 1:600, 1:550, 1:500, 1:450, 1:400, 1:350, 1:300, 1:250, 1:200, 1:150, 1:100, 1:50, 1:25, 1:10, or about 1:5.

### Manufacturing Process

In one embodiment the compositions disclosed herein are prepared by heating the cannabinoid, particularly if it is not already a liquid at room temperature, in order to increase its fluidity and/or reduce its viscosity. The surfactant is mixed with the cannabinoid and the mesoporous silica. The cannabinoid and the surfactant form a cannabinoid mixture that adsorbs to the mesoporous silica, that is the cannabinoid mixture is loaded into the mesoporous silica.

In an alternative embodiment the cannabinoid is heated as above and mixed with the surfactant to form a cannabinoid mixture. The cannabinoid mixture is then mixed with the mesoporous silica, wherein the cannabinoid mixture adsorbs to the mesoporous silica.

If the cannabinoid is a low-to-medium viscosity liquid at room temperature the heating step may not be required. However, typically cannabinoids exist as viscous oils, or in crystalline form at room temperature. In these cases the cannabinoid is heated to a temperature that increases the fluidity and/or decreases the viscosity of the cannabinoid in order to facilitate ease of handling. For example the cannabinoid can be heated to about 25° C., about 30° C., about 35° C., about 40° C., about 45° C., about 50° C., about 55° C., about 60° C., about 65° C., about 70° C., about 75° C., about 80° C., about 85° C., about 90° C., about 95° C., or about 100° C.

In embodiments where the cannabinoid is crystalline at room temperature, it is heated above its melting temperature. The cannabinoid may be heated to about 20° C. above its melting temperature. For example the cannabinoid may be heated to 5° C., 10° C., 15° C. or 20° C. above its melting temperature. The melting temperature of cannabinoids that are crystalline at room temperature are known in the art or can readily be determined by a skilled person.

In embodiments where the cannabinoid is resinous at room temperature, it is heated above its glass transition temperature. The cannabinoid may be heated to about 20° C. above its glass transition temperature. For example the cannabinoid may be heated to 5° C., 10° C., 15° C. or 20° C. above its glass transition temperature. The glass transition temperature of cannabinoids that are resinous at room temperature are known in the art, or can readily be determined by a skilled person.

The cannabinoid may be heated in the absence or in the presence of the surfactant. The process may further comprise the step of stirring the cannabinoid mixture and the mesoporous silica.

In some embodiments the cannabinoid mixture is primarily located in the pores of the mesoporous silica, with little or no mixture outside the pores. In other embodiments, the cannabinoid mixture is located outside the pores of the mesoporous silica. Both embodiments require the cannabinoid mixture to be loaded or adsorbed onto the mesoporous silica.

In embodiments where the composition is formulated into a dosage form, it is advantageous to minimize the size of the dosage form, and it is typically advantageous to maximize the drug loading. Given the challenges in loading high amounts of cannabinoid mixture into nano-sized pores, several loading techniques have been developed. The loading techniques require the cannabinoid mixture to be fluidized, either as a liquid solution or through heating (e.g., melt) as described above. Loading the mixture as a melt provides an advantage in that no subsequent evaporation step is necessary to remove the solvent medium.

During loading, one or more diluents may be incorporated (e.g., Medium Chain Triglyceride) when loading resinous cannabinoids with the melt method. The inventors have observed that viscosity of the cannabinoid or cannabinoid mixture has an influence on the desorption process. In many cases, melts of cannabinoids can still have high viscosities that hinder both drug loading and desorption. A diluent can be used to ‘thin’ the cannabinoid, making it easier to handle during formulation and can facilitate loading (i.e., flow) into the pores of the mesoporous silica. The diluent can also provide a competitive interaction between the silica surface and the cannabinoid or cannabinoid mixture during loading. Upon contact with the aqueous media, this diluent facilitates desorption and improves the extent of cannabinoid release, especially when combined with a solubilizer or emulsifier.

Various other methods may be used to load the cannabinoid mixtures described herein into mesoporous silica.

Solvent-based approaches may be used. These require a subsequent drying step to evaporate the solvent(s), which can be accomplished using many different available drying techniques that are well known to those skilled in art, including for example, use of a Rotavap (rotary evaporator). For example, this method can involve soaking mesoporous silica in a solution of cannabinoid mixture in a solvent, typically with stirring while preventing solvent evaporation. The solvent is then typically removed with a rotary evaporator.

In the heat method, the cannabinoid mixture and the mesoporous silica are heated to allow the mixture to become a liquid or to reduce the viscosity of the liquid. This is followed by mixing to load the cannabinoid mixture into the pores (i.e., allowing adsorption to occur). In some cases, a portion of cannabinoid mixture may also be loaded onto the external surface of the mesoporous silica.

In some embodiments, heating is not required. In these embodiments the cannabinoid mixture and mesoporous silica are combined at room temperature and the mixture is adsorbed to the mesoporous silica at room temperature.

An alternative method to load the cannabinoid mixture onto the mesoporous silica involves dissolving the cannabinoid or cannabinoid mixture in a liquid solvent medium before combining it with the mesoporous silica. The solvent can then be evaporated using any method known in the art such as evaporation or filtration. Similarly, in an incipient wetness impregnation approach, a concentrated solution of dissolved cannabinoid is mixed with mesoporous silica and the liquid is taken up through capillary forces. Using multiple cycles of loading and solvent evaporation, the cannabinoid mixture is loaded in multiple stages into the mesopores until the target theoretical load is achieved. This is a preferred method for crystalline cannabinoids because the majority of the solvent can be removed before the next loading cycle. In comparison, when this approach is used with resinous cannabinoids much of the solvent can remain in the pores making multiple loading cycles less effective.

Spray-drying can also be used to load the mesoporous silica and provide a composition of the invention. This process can be divided into four subprocesses: (1) feedstock preparation, (2) atomization, (3) drying, and (4) collection. The liquid feedstock consists of a suspension of mesoporous silica in a concentrated cannabinoid solution (see above). The resulting particle size and morphology can be fine-tuned according to the excipients and process parameters used.

Another loading method utilizes the fluidized bed approach in mixing, granulation (if required), and drying are all carried out in the same equipment. First, a suspension of a given cannabinoid-to-silica ratio is formulated and thoroughly mixed. The solvent in this suspension is then evaporated by spraying the suspension with the fluidized bed equipment.

Co-milling may also be used. In this solvent-free mechanical shearing process is reportedly disrupts the crystalline structure of a cannabinoid without causing significant chemical degradation through use of a low-energy jar-milling configuration. Physical mixtures of crystalline compounds (such as cannabinoids) and a mesoporous silica at suitable proportions are co-ground at room temperature. This leads to what is known as spontaneous amorphization in which the cannabinoid or cannabinoid mixture are adsorbed onto the mesoporous silica.

In the case of resinous cannabis material, cryogenic milling is a suitable method for adsorption onto mesoporous silica.

### Cannabinoid Release

The invention provides compositions that have enhanced or controlled release of the cannabinoid.

The release rate is influenced by a combination of the properties of the loaded cannabinoid mixture and the mesoporous silica, including pore diameter and pore morphology. The pore diameter of the mesoporous silica is an important factor affecting the release rate of the cannabinoid, with the release rate tending to increase as pore diameter increases. In addition to pore size, the pore morphology can also be modified in order to control the release rate of the cannabinoid. The particle size and shape affect the length of the pathway that a cannabinoid needs to diffuse in order to be released. For example, spherical SBA-15 particles have a larger number of pore openings compared to fiber-like particles. Pore length also influences the release rate and, in general, compositions that have delayed cannabinoid release comprise mesoporous silica having pores with a more tortuous diffusion route. This makes the deeper parts of the particle less accessible to a solvent and hence the selection of mesoporous silica is important for controlling the release profile of a cannabinoid. Accordingly, the release rate of the cannabinoid can be controlled by choice of mesoporous silica, in particular the pore size and pore geometry.

In some embodiments the compositions have enhanced cannabinoid release. The rate of dissolution of the cannabinoid from the mesoporous silica is related to the confined space inside the pores that prevents long range ordering, thus preventing the crystallization of the loaded substances. This stabilized amorphous form of the cannabinoid can improve its dissolution rate.

In particular, on contact with an aqueous release medium (such as a bodily fluid, stomach or intestinal contents), water penetrates the pores and the adsorbed hydrophobic cannabinoid mixture is displaced from the hydrophilic silica surface and transported by way of Fickian diffusion. The release rate depends on factors such as porosity, the cannabinoid's solubility in the release medium, the initial load, and the diffusion coefficient of the cannabinoid molecules in the medium.

### Formulations

The compositions disclosed herein may be formulated into any known dosage form. The formulations described herein may comprise one or more pharmaceutically acceptable excipients including carriers, vehicles and diluents. The term “excipient” herein means any substance, not itself an active agent, used as a diluent, adjuvant, or vehicle added to a formulation to improve its handling or storage properties or to permit or facilitate formation of a solid dosage form such as a tablet, capsule, or a solution or suspension suitable for oral, parenteral, intradermal, subcutaneous, or topical application. Excipients can include, by way of illustration and not limitation, diluents, disintegrants, binding agents, adhesives, wetting agents, polymers, lubricants, glidants, stabilizers, and substances added to mask or counteract a disagreeable taste or odor, flavors, dyes, fragrances, and substances added to improve appearance of the composition. Acceptable excipients include (but are not limited to) stearic acid, magnesium stearate, magnesium oxide, sodium and calcium salts of phosphoric and sulfuric acids, magnesium carbonate, talc, gelatin, acacia gum, sodium alginate, pectin, dextrin, mannitol, sorbitol, lactose, sucrose, starches, gelatin, cellulosic materials, such as cellulose esters of alkanoic acids and cellulose alkyl esters, low melting wax, cocoa butter or powder, polymers such as polyvinyl-pyrrolidone, polyvinyl alcohol, and polyethylene glycols, and other pharmaceutically acceptable materials. Examples of excipients and their use is described in Remington's Pharmaceutical Sciences, 20th Edition (Lippincott Williams & Wilkins, 2000). The choice of excipient will to a large extent depend on factors such as the particular mode of administration, the effect of the excipient on solubility and stability, and the nature of the dosage form.

The formulations of the invention are suitable for oral, rectal, vaginal, or topical delivery. Non-limiting examples of particular formulation types include tablets, troches, capsules, caplets, powders, granules, ready-to-use solutions or suspensions, lyophilized materials, gels, creams, lotions, ointments, drops, and suppositories. Solid formulations such as the tablets or capsules may contain any number of suitable pharmaceutically acceptable excipients or carriers described above.

Tablets and capsules for oral administration may be in unit dose presentation form, and may contain conventional excipients such as binding agents, for example, acacia, gelatin, sorbitol, tragacanth, or polyvinylpyrrolidone; fillers, for example lactose, sugar, maize-starch, calcium phosphate, sorbitol or glycine; tabletting lubricants, for example, magnesium stearate, talc, polyethylene glycol or silica; disintegrants, for example, potato starch; or acceptable wetting agents such as sodium lauryl sulphate. The tablets may be coated according to methods well known in pharmaceutical practice.

Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives, such as suspending agents, for example, sorbitol, methyl cellulose, glucose syrup, gelatin, hydroxyethyl cellulose, carboxymethyl cellulose, aluminium stearate gel or hydrogenated edible fats, emulsifying agents, for example, lecithin, sorbitan monooleate, or acacia; non-aqueous vehicles (which may include edible oils), for example, almond oil, oily esters such as glycerin, propylene glycol, or ethyl alcohol; preservatives, for example, methyl or propyl p-hydroxybenzoate or sorbic acid; and, if desired, conventional flavouring or colouring agents.

The effective amount of the cannabinoid in the formulation that is administered and the dosage regimen with the compositions and/or formulations of the present invention depends on a variety of factors, including the age, weight, sex, and medical condition of the subject, the severity of the disease, the route and frequency of administration, the particular compound employed, as well as the pharmacokinetic properties (e.g., adsorption, distribution, metabolism, excretion) of the individual treated, and thus may vary widely. Such treatments may be administered as often as necessary and for the period of time judged necessary by the treating physician or other medical professional. One of skill in the art will appreciate that the dosage regimen or therapeutically effective amount of the compound to be administrated may need to be optimized for each individual.

The compositions may contain active ingredient in the range of about 0.1 mg to 2000 mg, typically in the range of about 0.5 mg to 500 mg and more typically between about 1 mg and 200 mg. A daily dose of about 0.01 mg/kg to 100 mg/kg body weight, typically between about 0.1 mg/kg and about 50 mg/kg body weight, may be appropriate, depending on the route and frequency of administration.

In one embodiment, the formulations are consumed orally. A single dose is at from 0.1 mg but may be up to about 250 mg. For example a single dose may be 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 190 mg, 195 mg, 200 mg, 205 mg, 210 mg, 215 mg, 220 mg, 225 mg, 230 mg, 235 mg, 240 mg, 245 mg, 250 mg, 275 mg, 300 mg, 325 mg, 350 mg, 400 mg, 450 mg, 475 mg, or at least about 500 mg.

A subject may consume one or multiple doses per day. For example, a subject may take 1, 2, 3, 4 or 5 doses per day. In some embodiments, the dosing interval is selected from the group consisting of once per week dosing, twice per week dosing, three times per week dosing, four times per week dosing, five times per week dosing, six times per week dosing, weekly dosing, and twice-monthly dosing. In other embodiments, dosing may be as needed or as desired by the user.

### Pharmaceutical Use

The compositions and formulations described herein contain cannabinoids and the invention also relates to a method of treating a condition or disease responsive to a cannabinoids such as pain (including chronic pain), spasticity associated with multiple sclerosis, nausea (chemotherapy-induced nausea and vomiting), posttraumatic stress disorder, cancer, epilepsy, cachexia, glaucoma, HIV/AIDS, degenerative neurological conditions, anorexia and weight loss associated with HIV, irritable bowel syndrome, epilepsy, spasticity, Tourette syndrome, amyotrophic lateral sclerosis, Huntington's disease, Parkinson's disease, dystonia, dementia, traumatic brain injury, addiction, anxiety, depression, sleep disorders, and schizophrenia and other psychoses.

A further embodiment relates to the use of the compositions disclosed herein for the manufacture of a medicament for treating a disease or condition responsive to a cannabinoid, such as those listed above.

The compositions of the present invention may be administered along with a pharmaceutical carrier, diluent or excipient as described above. Alternatively, or in addition, the compounds may be administered in combination with other agents, for example, other therapeutic agents.

The terms “combination therapy” or “adjunct therapy” in defining use of a compound of the present invention and one or more other pharmaceutical agents, are intended to embrace administration of each agent in a sequential manner in a regimen that will provide beneficial effects of the drug combination, and is intended as well to embrace co-administration of these agents in a substantially simultaneous manner, such as in a single formulation having a fixed ratio of these active agents, or in multiple, separate formulations of each agent.

In accordance with various embodiments of the present invention, the composition may be formulated or administered in combination with one or more other therapeutic agents. Thus, in accordance with various embodiments of the present invention, a composition may be included in combination treatment regimens with known treatments or therapeutic agents, and/or adjuvant or prophylactic agents.

Combination regimens may involve the active agents being administered together, sequentially, or spaced apart as appropriate in each case. Combinations of active agents including compounds of the invention may be synergistic.

For example, the composition may further comprise pharmacologically active agents that are poorly soluble in water. Examples of suitable agents include:
anesthetics such as bupivacaine, lidocaine, proparacaine, and tetracaine; analgesics, such as acetaminophen, ibuprofen, fluriprofen, ketoprofen, voltaren, phenacetin, and salicylamide;
anti-inflammatories selected from the group consisting of naproxen and indomethacin;
antihistamines, such as chlorpheniramine maleate, phenindamine tartrate, pyrilamine maleate, doxylamine succinate, phenyltoloxamine citrate, diphenhydramine hydrochloride, promethazine, brompheniramine maleate, dexbrompheniramine maleate, clemastine fumarate and triprolidine;
broad and medium spectrum, antimicrobial agents such as erythromycin, penicillin and cephalosporins and their derivatives;
Skeletal muscle relaxants (dantrolene sodium, baclofen), benzodiazepines (diazepam), alpha2-adrenergic agonists (clonidine, tizanidine), botulinum toxins (onabotulinumtoxinA, abobotulinumtoxinA, incobotulinumtoxinA, rimabotulinumtoxinB);
5-HT₃ inhibitors such as dolasteron (Anzemet), granisetron (Kytril, Sancuso), and ondansetron (Zofran) palonosetron (Aloxi));
NK1 inhibitors (e.g., substance P inhibitor aprepitant (Emend), Netupitant, Rolapitant;
Olanzapine;
A combination of palonosetron and dexamethasone;
Dopamine D2 receptor antagonist e.g., Metoclopramide;
Histamine blockers such as diphenhydramine or meclozine;
acetazolamide, carbamazepine, clobazam clonazepam, diazepam, ethosuximide, fosphenytoin, gabapentin, lacosamide, lamotrigine, levetiracetam, lorazepam, methsuximide, nitrazepam, oxcarbazepine, paraldehyde, phenobarbital, phenytoin, pregabalin, primidone, rufinamide, stiripentol, topiramate, valproic acid, vigabatrin, felbamate, tiagabine hydrochloride, zonisamide Lorazepam, diazepam
Progestagens such as megestrol acetate and medroxyprogesterone acetate,
Omega-3 fatty acids (e.g., EPA)
bortezomib
thalidomide
ghrelin
COX-2 inhibitors
branched chain amino acids
oxandrolone
alpha-adrenergic agonists
carbonic anhydrase inhibitors
parasympathomimetics
Anti-retrovirals
Fluphenazine, haloperidol (Haldol), risperidone (Risperdal) and pimozide (Orap); quetiapine
Riluzole (Rilutek), Edaravone (Radicava)
Tetrabenazine, amantadine, levetiracetam.

The co-administration of compounds of the invention may be effected by the compounds being in the same unit dose as another active agent, or the compounds and one or more other active agent(s) may be present in individual and discrete unit doses administered at the same, or at a similar time, or at different times according to a dosing regimen or schedule. Sequential administration may be in any order as required, and may require an ongoing physiological effect of the first or initial compound to be current when the second or later compound is administered, especially where a cumulative or synergistic effect is desired.

### Consumer Products

In other embodiments the compositions may be included in consumer products such as food products, cosmetics, and sunscreen.

The food product may be a baked good (for example a bread, cake, biscuit or cookie, beverage (e.g., tea, soda or flavored milk), breakfast food (e.g., cereal), muesli bar, tinned food, snack food (e.g., chips, crisps, corn snacks, nuts, seeds), confection, condiment, marinade, dairy product, dips, spreads or soups.

The cosmetic may be a be liquid, lotion, cream, powder (pressed or loose), a dispersion, an anhydrous cream or stick. For example, the cosmetic may be a spray, perfume, foundation, mascara, lipstick, lip gloss, lip liner, lip plumper, lip balm, lip stain, lip conditioner, lip primer, lip booster, lip butter, deodorant, bath oils, bubble baths, bath salts, body butter, nail polish, hand sanitizer, shampoo, conditioner, hair colors, hair sprays, hair gels, primer, concealer, highlighter, bronzer, mascara, eye shadow, eyebrow pencils, eyebrow cream, eyebrow wax, eyebrow gel, eyebrow powder, moisturizer, or toner.

The consumer product may contain less than about 1% (w/w) of the composition or about 1% (w/w), or about 2% (w/w), or about 3% (w/w), or about 4% (w/w), or about 5% (w/w), or about 6% (w/w), or about 7% (w/w), or about 10% (w/w), or about 11% (w/w), or about 12% (w/w), or about 13% (w/w), or about 14% (w/w), or about 15% (w/w), or about 16% (w/w), or about 17% (w/w), or about 18% (w/w), or about 19% (w/w), or about 20% (w/w), or about 25% (w/w), or about 30% (w/w), or about 35% (w/w), or about 40% (w/w), or about 45% (w/w), or about 50% (w/w) of the composition.

It will be appreciated by persons skilled in the art that numerous variations and/or modifications may be made to the technology as shown in the specific embodiments without departing from the spirit or scope of technology as broadly described. The present embodiments are, therefore, to be considered in all respects as illustrative and not restrictive.

### EXAMPLES

### Example 1: Delta-9-THC Mesoporous Silica Composition

Syloid 3050 XDP (50 μm median particle size) was selected as the mesoporous silica carrier and sodium lauryl sulfate (SLS) as the anionic surfactant. A delta-9-THC distillate with 85% purity, as determined by HPLC, was selected for method development and optimization purposes. To improve the ease of handling, the viscosity of the distillate was decreased by placing it in a 40° C. oven for approximately 15 minutes prior to weighing. Following removal from the oven, it was immediately added to the pre-weighed Syloid 3050 XDP and SLS to a target drug load of 35-40% (w/w) delta-9-THC (˜40-45% cannabis extract) and 4% (w/w) SLS. The mixture was blended using a standard overhead laboratory mixer until all of the distillate visually appeared to be adsorbed onto the silica. The dry powder blend was then sieved through a 60 μm sieve.

The potency of the drug loaded silica/SLS mixture was then analyzed by HPLC-UV by diluting samples to ˜100 μg/mL in acetonitrile (ACN) and analyzed (n=3 replicates) at room temperature. All standard curves were linear over the concentration range of 0.7-700 μg/mL. The measured potency was evaluated against the theoretical potency with an acceptance criterion set to ≤10% (i.e., 90-100% of the theoretical maximum loading capacity). This resulting potency was then used to determine the target weight to achieve a 25 mg delta-9-THC dose in a 175 mg tablet (14.3% w/w).

### Example 2: Immediate Release Oral Tablet (Delta-9-THC)

The tablet blend was prepared (see example below) using an optimized blending procedure and analyzed using HPLC for potency and homogeneity with acceptance criteria of ±5% for both. Following this, tablets were prepared by direct compression using standard tableting excipients. Tablets were also analyzed for potency and batch homogeneity using HPLC. Finally, the tablet weight, thickness, and hardness were also assessed and compared against the batch release specifications prior to sale.

TABLE 3
delta-9-THC ingredients

| Material | Function | Weight (%) |
|---|---|---|
| d9-THC distillate (~85% purity)* | Active | 14.3 |
| Syloid 3050 XDP* (mesoporous silica) | Adsorbent | 43.0 |
| Sodium Lauryl Sulfate* | Surfactant | 1.5 |
| Avicel PH-101 (microcrystalline cellulose) | Binder | 35.0 |
| Ac-Di-Sol (croscarmellose sodium) | Disintegrant | 5.0 |
| Mg Stearate | Lubricant | 1.0 |
| Terpenoids (cannabis derived, steam distilled) | Active | 0.2 |

| | |
|---|---|
| Total | 100.0 |

*Combined during drug loading step and analyzed prior to blending/compression.

In vitro observations were consistent with the above findings during development with delta-8-THC and delta-9-THC distillate. The resulting HPLC potency was consistently 25-30% below the theoretical loading. This is attributed to insufficient desorption from the silica surface. However, this was mitigated through the use of sodium lauryl sulfate, a surface active agent (“surfactant”).

The incorporation of 1.5% SLS to the drug loaded silica improved the potency to within 10% of the theoretical value. Follow-up investigations evaluating the influence of higher surfactant concentrations (up to 10% SLS) did not improve delivery.

### Example 3: CBD Mesoporous Silica Tablets

The tablet blend was prepared in accordance with table 4 and tablets were prepared by direct compression

TABLE 4
CBD ingredients

| Material | Function | Weight (%) |
|---|---|---|
| CBD (76.5% purity) | Active | 2.6 |
| Syloid 244 (mesoporous silica) | Adsorbent | 1.0 |
| Xylitol | Filler/binder | 19.4 |
| Orange oil | Flavour | 1.3 |
| Spearmint oil | Flavour | 2.7 |
| Ac-Di-Sol (croscarmellose sodium) | Disintegrant | 1.5 |
| Kollidon ® (Crospovidone) | Disintegrant | 5.0 |
| Ludiflash ® | Filler/binder/disintegrant | 65.5 |
| Mg Stearate | Lubricant | 1.0 |

| | |
|---|---|
| Total | 100.0 |

### Example 4: CBD Mesoporous Silica Tablets

The tablet blend was prepared in accordance with table 5 and tablets were prepared by direct compression

TABLE 5
CBD ingredients

| Material | Function | Weight (%) |
|---|---|---|
| CBD (76.5% purity) | Active | 12.9 |
| Aerosil (mesoporous silica) | Glidant | 1.7 |
| Ac-Di-Sol (croscarmellose sodium) | Disintegrant | 4.8 |
| Microcrystalline cellulose | Filler/binder/disintegrant | 79.4 |
| Mg Stearate | Lubricant | 1.2 |

| | |
|---|---|
| Total | 100.0 |

### Example 5: Incipient Wetness Loading of Resinous Distillate onto Mesoporous Silica

Resinous distillate was dissolved in 99% isopropanol (iso-propyl alcohol) to a concentration of 147.25 mg/ml. A portion of this solution was added to the mesoporous silica (either Syloid 3050 XDP or Syloid 244). The portion of the solution added to the silica is equal or slightly less than the pore volume of the silica. After the cannabinoid is adsorbed onto the silica the isopropanol is removed by evaporation and another portion of the solution is added to silica. This process is repeated until the desired amount of distillate (30% by weight of silica) was added.

### Example 6: Incipient Wetness Loading of Delta-9-THC onto Mesoporous Silica

In this example a solution of 113.9 mg/ml Delta-9-THC in 99% isopropanol was prepared. A mixture of Syloid 244 and Syloid 3050 XDP was prepared in a 1:3 ratio., specifically 157 mg of Syloid 244 was mixed with 471 mg of Syloid 3050 XDP (total weight of 628 mg). 0.2 ml of the Delta-9-THC solution was added incrementally to the silica mixture until a total of 6 ml of the solution was added, for a total of 683.4 mg Delta-9-THC. After the last of the solvent was removed by evaporation the weight of the silica had increased to 1311.4 mg indicating that all of the Delta-9-THC was loaded onto the silica and the total load of Delta-9-THC was 52%. The loaded silica was a flowable powder.

When this test was repeated the total load of Delta-9-THC was 49%. Again, the loaded silica was a flowable powder.

The test was repeated using Delta-9-THC in isopropanol and 1:4 and 1:5 mixtures of Syloid 244:Syloid 3050 XDP. Although it was found that at these ratios the total load of Delta-9-THC was slightly lower (44%), these mixtures had more desirable flow characteristics than the 1:3 mixture of Syloid 244:Syloid 3050. Syloid 3050 (150 um particle size) flows very well in comparison to Syloid 244 (2 um particle size). The incorporation of 244 can create harder tablets due to filling of the powder blend ‘voids’. Syloid 244 is also more suitable for crystalline cannabinoids.

### Example 7: Scale-Up Incipient Wetness Loading of Resinous Distillate onto Mesoporous Silica

In this example a 1:4 mixture of Syloid 244:Syloid 3050 was prepared using 3.19 g Syloid 244 and 12.32 g Syloid 3050 (a total of 15.51 g silica).

A solution of 60.36 mg/ml resinous distillate in isopropanol was prepared by first softening the distillate by hearting then adding the isopropanol.

The silica mixture was separated into three portions and the volume of the resinous distillate solution required to achieve 25%, 37.5% and 50% loading of the silica was calculated.

As for previous examples the silica mixture was loaded by incrementally adding the aliquots of the solution of resinous distillate to the silica. Solvent was removed using a rotary evaporator (Rotovap®) before the addition of more solution.

### Example 8: Delta-9-THC Tablets

The tablet blend was prepared in accordance with table 6 and tablets were prepared by direct compression

TABLE 6
Ingredients

| Material | Function | Weight (%) |
|---|---|---|
| Loaded silica (from example 6. 1:4 | Active | 42.2 |
| mixtures of Syloid 244:Syloid 3050 | | |
| loaded with Delta-9-THC). | | |
| Ac-Di-Sol (croscarmellose sodium) | Disintegrant | 4.1 |
| Microcrystalline cellulose | Filler/binder/disintegrant | 52.4 |
| Mg Stearate | Lubricant | 1.3 |
| Total | | 100.0 |

### Example 9: CBG Tablets

The tablet blend was prepared in accordance with table 7 and tablets were prepared by direct compression

TABLE 7
Ingredients

| Material | Function | Weight (%) |
|---|---|---|
| CBG (98.3% purity) | Active | 17.0 |
| Aerosil (mesoporous silica) | Glidant | 1.6 |
| Ac-Di-Sol (croscarmellose sodium) | Disintegrant | 4.7 |
| Microcystalline cellulose | Filler/binder/disintegrant | 75.6 |
| Mg Stearate | Lubricant | 1.1 |

| | |
|---|---|
| Total | 100.0 |

### Example 10: Controlled Release Delta-9-THC Tablets

A 1:4 mixture of Syloid 244:Syloid 3050 was loaded with delta-9-THC as described in Example 6. Once loaded the dried silica mixture contained 40% delta-9-THC by weight. This loaded silica was used to prepare the tablet blend in accordance with table 8. Following this, tablets were prepared by direct compression.

TABLE 8
Ingredients

| Material | Function | Weight (%) |
|---|---|---|
| Loaded Silica | Active | 26.8 |
| Hydroxypropyl methylcellulose | Controlled release agent | 11.5 |
| Ac-Di-Sol (croscarmellose sodium) | Disintegrant | 6.4 |
| Microcystalline cellulose | Filler/binder/disintegrant | 54 |
| Mg Stearate | Lubricant | 1.3 |

| | |
|---|---|
| Total | 100.0 |

### Example 11: HPLC Method to Quantitate Cannabinoids

It was found that the Luna Omega C18 HPLC column provides resolution for cannabinoids under the following conditions:
mobile phase: 5 mM ammonium acetate (pH 4.5 with acetic acid) in 80:20, acetonitrile:water
flow rate: 1.0 mL/min
pressure: 2000 psi (138 bar)
column temp.: room temperature
detector: UV, 214 nm

Cannabinoids were eluted from the column and identified by reference to known controls eluted from the column under the same conditions. Cannabinoids were quantitated by reference to a calibration curve of peak area vs concentration of the known controls.

The relative standard deviation (% RSD) for measurements of THC and CBD using this method is 3.2% (n=5) and 0.7% (n=3), respectively. Recovery is greater than 95%.

### Example 12: Cannabinoid Release from Loaded Silica

A silica blend loaded with 37.5% delta-9-THC was prepared as per Example 7. Samples of the blend containing a calculated 7.5 mg of delta-9-THC were mixed with 900 μL of acetonitrile. HPLC was performed according to Example 11 and it was found that the amount of delta-9-THC recovered from the silica blend was 23.54% (RSD of 6.4%, n=3).

Similarly, when 220 mg aliquots of the blend were extracted with methanol overnight before HPLC recovery of delta-9-THC, around 80% of the total cannabinoid loaded (RSD of 0.9%, n=3).

### Example 13: Inclusion of Surfactant Increases Cannabinoid Release from Loaded Silica

1.5% (w/w) sodium lauryl sulfate was added to a silica blend loaded with 37.5% delta-9-THC prepared as per Example 7 before mixing with acetonitrile in accordance with Example 12. HPLC was performed according to Example 11 and it was found that the amount of delta-9-THC recovered from the silica blend was on average 33.9% (n=3), i.e., about 90% of the cannabinoid loaded onto the silica was recovered.

When this test was repeated using a 1.5% (w/w) sodium lauryl sulfate added to a Syloid 3050 silica loaded with 37.5% delta-9-THC prepared as per Example 7 it was found that 94.76% of delta-9-THC loaded was recovered.

When the sodium lauryl sulfate was mixed with the cannabinoid prior to loading (see Examples 1 and 2) the recovery of delta-9-THC was within 10% of the calculated amount that was loaded.

1. A powder composition comprising a cannabinoid mixture adsorbed onto at least one mesoporous silica wherein the cannabinoid mixture comprises a cannabinoid and a surfactant to facilitate desorption of the cannabinoid from the mesoporous silica.

2. The composition of claim 1, wherein the cannabinoid is selected from the group consisting of a plant extract, cannabigerolic acid (CBGA); cannabigerolic acid monomethylether (CBGAM), cannabigerol (CBG), cannabigerol monomethylether (CBGM), cannabigerovarinic acid (CBGVA), cannabichromevarin (CBCV), cannabichromenic acid (CBCA) cannabichromene (CBC), cannabidiolic acid (CBDA), cannabidiol (CBD), cannabidiol monomethyl ether (CBDM), cannabidiol-C4 (CBD-D4), cannabidivarinic acid (CBDVA), cannabidivarin (CBDV), cannabidiorcol (CBD-D1), delta-9-tetrahydrocannabinolic acid A (THCA-A), delta-9-tetrahydrocannabinolic acid B (THCA-B), delta-9-tetrahydrocannabinol (D9-THC), 11-hydroxy-delta-9-tetrahydrocannabinol (11-OH-THC), delta-9-tetrahydrocannabinolic acid C4 (THCA-C4), delta-9-tetrahydrocannabinol-C4 (THC-C4), delta-9-tetrahydrocannabivarinic acid (THCVA), delta-9-tetrahydrocannabivarin (THCV), delta-9-tetrahydrocannabiorcolic acid (THCA-C1),), delta-9-tetrahydrocannabiorcol (THC-C1), delta-7-cis-iso-tetrahydrocannabivarin (D7-THCV), delta-8-tetrahydrocannabinolic (D8-THCA), delta-8-tetrahydrocannabinol (D8-THC), cannabicycloic acid (CBLA), cannabicyclol (CBL), cannabicyclovairn (CBLV), cannabielsoic acid A (CBEA-A), cannabielsoic acid B (CBEA-B), cannabielsoin (CBE), cannabinolic acid (CBNA), cannabinol (CBN), cannabinol methylether (CBNM), cannabinol-C4 (CBN-C4), cannabinol-C2 (CBN-C2), cannabivarin (CBV), cannabiorcol (CBN-C1), cannabinodiol (CBND), cannabinodivarin (CBVD), cannabitriol (CBT), 10-ethoxy-9-hydroxy-delta-6a-tetrahydrocannabinol, 8,9-dihydroxy-delta-6a-tetrahydrocannabinol, cannabitriolvarin (CBTV), ethoxy-cannabitriolvarin (CBTVE), dehydrocannabifuran (DCBG), cannabifuran (CBF), cannabichromanon (CBCN), cannabicitran (CBT), 10-oxo-delta-6a-tetrahydrocannabinol (OTHC), delta-9-cis-tetrahydrocannabinol (cis-THC), 3,4,5,6-tetrahydro-7-hydroxy-alpha-alpha-2-trimethyl-9-n-propyl-2,6-methano-2H-1-benzoxoxin-5-methanol (OH-iso-HHCV), cannabiripsol (CBR), and trihydroxy-delta-9-tetrahydrocannabinol (triOH-THC).

3. The composition of claim 1, wherein the cannabinoid is Delta-9-THC or Delta-8-THC.

4. The composition of claim 1, wherein the surfactant is an anionic, cationic, or zwitterionic surfactant.

5. The composition of claim 4, wherein the anionic surfactant is sodium lauryl sulfate.

6. The composition of claim 1, wherein the surfactant:cannabinoid mass ratio is from about 1:1000 to about 1:5.

7. The composition of claim 1, wherein the composition further comprises a terpene or terpenoid.

8. The composition of claim 1, wherein the cannabinoid mixture further comprises a diluent.

9. The composition of claim 1, wherein the mass ratio of diluent:cannabinoid is between about 1:50 to about 50:1.

10. The composition of claim 9, wherein the diluent is a plant or vegetable oil.

11. The composition of claim 1, wherein the mesoporous silica is ordered mesoporous silica or disordered mesoporous silica.

12. The composition of claim 1, wherein the mesoporous silica has an average pore volume of about 0.5 cm³/g to about 10 cm³/g.

13. The composition of claim 1, wherein the mesoporous silica has an average pore size of about 2 nm to about 50 nm.

14. The composition of claim 1, wherein the mesoporous silica are mesoporous silica particles.

15. The composition of claim 14, wherein the particles have an average diameter of about 2 μm to at least about 250 μm, for example the average diameter may be about 2 μm, about 10 μm, about 25 μm, about 50 μm, about 75 μm, about 100 μm, about 125 μm, about 150 μm, about 175 μm, about 200 μm, about 225 μm, or at least about 250 μm.

16. The composition of claim 1, wherein the mass ratio of surfactant:mesoporous silica is from about 1:50 to about 1:5.

17. The composition of claim 16, wherein the mass ratio of surfactant:mesoporous silica is from about 1:35 to about 1:25 or about 1:29.

18. The composition of claim 1, wherein the composition is a flowable powder.

19. The composition of claim 1, wherein the composition comprises a blend of two or more mesoporous silica.

20. A food, beverage, cosmetic product or formulation comprising the composition of claim 1.

21. The formulation of claim 20, further comprising at least one excipient, vehicle, or diluent.

22. The formulation of claim 21, wherein the excipient is one or more of microcrystalline cellulose, croscarmellose sodium, and magnesium stearate.

23. A process of preparing the composition of claim 1, comprising
a) heating the cannabinoid;
b) mixing the cannabinoid with the surfactant wherein the cannabinoid and the surfactant form a cannabinoid mixture;
c) mixing the cannabinoid mixture with the mesoporous silica, wherein the cannabinoid mixture adsorbs to the mesoporous silica.

24. (canceled)

25. A method of treatment of a disease or condition, the method comprising administering to the subject an effective amount of a composition of claim 1, wherein the disease or condition is selected from the group consisting of pain, spasticity associated with multiple sclerosis, nausea, posttraumatic stress disorder, cancer, epilepsy, cachexia, glaucoma, HIV/AIDS, degenerative neurological conditions, anorexia and weight loss associated with HIV, irritable bowel syndrome, epilepsy, spasticity, Tourette syndrome, amyotrophic lateral sclerosis, Huntington's disease, Parkinson's disease, dystonia, dementia, traumatic brain injury, addiction, anxiety, depression, sleep disorders, and schizophrenia.

26. The composition of claim 1, wherein the mesoporous silica has a specific surface area of about 700 m²/g to about 1,000 m²/g.
